(19) United States
(12) Patent Application Publication
(10) Pub. No.: US 2022/0079974 A1
(43) Pub. Date: Mar. 17, 2022

(54) A COMPOSITION FOR THE TREATMENT OF PERIODONTITIS AND REGENERATION OF INTERDENTAL PAPILLA

(71) Applicant: MASTELLI S.R.L., Sanremo (Imperia) (IT)

(72) Inventors: Giovanni Prussia, Sanremo (Imperia) (IT); Claudia Prussia, Sanremo (Imperia) (IT); Giulia Cattarini Mastelli, Sanremo (Imperia) (IT)

(73) Assignee: MASTELLI S.R.L., Sanremo (Imperia) (IT)

(21) Appl. No.: 17/425,012

(22) PCT Filed: Jan. 24, 2020
(86) PCT No.: PCT/IB2020/050567

§ 371 Date: Jul. 22, 2021

(30) Foreign Application Priority Data
Jan. 24, 2019 (IT) 102019000001081

(51) Int. Cl. A61K 31/728; A61K 31/7088; A61P 1/02

(52) U.S. Cl. CPC A61K 31/728; A61P 1/02; A61K 31/7088

(57) ABSTRACT

A composition comprising hyaluronic acid and a mixture of polynucleotides extracted from a natural source, for use as an active substance in the therapeutic and/or cosmetic treatment for regeneration of the interdental papilla or in the therapeutic and/or cosmetic treatment of the recession of the interincisal papilla (black triangle syndrome) or in the therapeutic and/or cosmetic treatment of periodontitis, is described herein.

The present invention refers to a composition active in the therapeutic and/or cosmetic treatment of periodontitis and the regeneration of the interdental papilla.

Periodontitis is a disease characterised by inflammation of the periodontal tissues. It is one of the most prevalent chronic inflammatory diseases in the population of Western countries: in Italy, it is estimated to affect, in its severe form, about 10-15% of adults, while a percentage between 2 and 30% is estimated to be affected by a mild form thereof. Periodontitis is the main cause of dental element loss; it is responsible for a serious functional deficit and at the same time can negatively affect relationships, with a significant impact on the psychological sphere, compromising the smile aesthetics.

The characteristic sign of periodontitis is the loss of attachment of the teeth to the alveoli, which results in formation of periodontal pockets, loose teeth, bleeding gums, abscesses and suppurations, ending in the loss of one or more teeth.

Conventionally, treatment of periodontitis first of all involves controlling bacteria and inflammation, and immediately afterwards, the subsequent step of regenerating the lost, hard and soft periodontal tissues becomes essential, in order to restore tooth attachment and eliminate gingival and bone pockets.

The interdental papilla is the portion of the free gingiva that occupies the space between two adjacent teeth and causes the free gingival margin to run in the dental arches with a festooned pattern. The papilla, in addition to having significance in the biology and coating of the proximal portion of the teeth, has an important aesthetic significance especially in the front regions. Explicit attention to the preservation of all interproximal tissues for aesthetic purposes began with the 1985 article by Evian and co-workers (Evian C I, Corn H, Rosenberg E S. Retained interdental papilla procedure for maintaining anterior esthetics. Compend Contin Educ Dent 1985; 6(1):58-64), which describes a surgical method with specific aesthetic purposes, defining it as a “retained interdental papilla procedure”.

The loss of this portion of gingival tissue is also the subject of studies, in particular with reference to possible conservation and regeneration techniques. Dental aesthetics, in fact, has become increasingly of paramount importance, both for dentists and patients. The presence of interproximal papillae between the anterior maxillary teeth is, in fact, a key aesthetic component. The recession of the interdental papilla causes a number of functional problems, as well as aesthetic problems, such as the formation of the so-called black triangle (“black triangle syndrome” (or “recession of the interincisal papilla”).

In 1998, Nordland and Tarnow classified the loss of papillary height using a scale, which is still widely used and identifies four classes, namely Normal, Class I, Class II and Class III (Nordland W P, Tarnow D P. A classification sistem for loss of papillary heiht. J Periodontol 1998; 69:1124-6). The classes identified by Nordland and Tarnow use the following anatomical references:

1) interdental contact point;

2) apical/facial extension of the CEJ (Cementum Enamel Junction);

3) coronal/proximal extension of the CEJ.

These classes are briefly described below:
Normal: the papilla fills the interdental space up to the contact point.
Class I: the tip of the papilla is located between the contact point and the coronal part of the CEJ (the space is present but the coronal/proximal CEJ is not visible).
Class II: the tip of the papilla is located apical to the coronal/proximal CEJ, but level with the facial CEJ (visible coronal/proximal CEJ).
Class III: the tip of the papilla is located apical to the facial CEJ.

When interdental tissue loss has already occurred, numerous techniques with reconstructive purposes can be implemented. Methods proposed in the literature suitable for solving this tissue deficiency can be divided, in all, into surgical and non-surgical techniques, even though this distinction is merely descriptive since, frequently, surgical and non-surgical techniques can be combined with each other.

Surgical Reconstruction Techniques

Numerous surgical stratagems have been described in the literature with the purpose of correcting interdental tissue deficiency. However, the cited knowledge is often limited to case studies.

In 1992, Beagle proposed a surgical method, called the pedunculated flap technique, in order to recreate the interdental papilla between the upper central incisors (Beagle J R. Surgical reconstruction of the interdental papilla: case report. Int J Periodontics Restorative Dent 1992; 12(2):145-51). The applicability of this technique is limited to large interdental areas and possibly facilitated by thick biotypes due to a greater presence of connective tissue.

In 1994, Aubert and co-workers proposed the deep rotated connective tissue flap method, seeking greater conservation of the nutritional supply to the tissues involved (Aubert H, Bertrand G, Orlando S, Benguigui F, Acocella G. Lembo connettivale profondo di rotazione per ricostruire la papilla interdentale. Minerva Stomatologica 1994; 43 (7-8):351-357). However, this technique has the disadvantage that it can be used for one papilla at a time.

Still based on the intuition of the usefulness of a connective tissue filler, but with a view of simplifying the surgical technicality, in 1996 Han and Takei proposed the semilunar coronally repositioned papilla technique (Han T J, Takei H H. Progress in gingival papilla reconstruction. Periodontol 2000 1996; 11:65-8). This method is inspired by the consolidated reliability of the bilaminar technique, consisting in the use of a connective tissue graft underneath a partial thickness flap. One of the advantages of this technique is the dual blood supply from both the connective tissue and the overlying flap, which favours graft survival (Langer B, Langer L. Subepithelial connective tissue graft technique for root coverage. J Periodontol 1985; 56(12):715-20). Despite the intuition that the use of a bilaminar technicality leads to practical advantages, compared to the method previously proposed by Aubert et al., 1994, the coronal shift of the interdental tissue complex, introduced by Han and Takei, still represents an actual technical difficulty.

In 1998, Azzi et al. proposed the envelope technique, an approach similar to that previously described, but with some changes that potentially improve its applicability (Azzi R, Etienne D, Carranza F. Surgical reconstruction of the interdental papilla. Int J Periodontics Restorative Dent. 1998; 18(5):466-73).

Finally, the therapeutic difficulties associated with a significant tissue deficiency and the search for long-term stability led Azzi et al. to propose, in 2001, the use of an autologous bone graft (Azzi R, Takei H H, Etienne D, Carranza F A. Root coverage and papilla reconstruction using autogenous osseous and connective tissue grafts. Int J Periodontics Restorative Dent 2001; 21(2): 141-7).

In general, the aforementioned surgical reconstruction therapies create an aesthetic-anatomical compromise, altering the physiological relationship between soft and hard periodontal tissues. The clinical consequence is that a periodontal pocket or, at best, a long junctional epithelium is formed between the dental elements and the new interdental tissue. At the same time, the disruption of the normal relationship between the tissues leads to high uncertainty of the outcome in the long term.

Non-Surgical Reconstruction Techniques

Non-surgical reconstruction techniques are basically aimed at modifying non-gingival anatomical determinants related to the presence of interdental tissues. Therefore, their use makes it possible to act on the interdental space and/or on the distance between the contact point and the bone crest. Three types of approaches are suggested for these techniques:
Restorative/prosthetic
Orthodontic
With curettage and injection of fibroblasts

The potential of the restorative/prosthetic treatment consists in modifying the coronal morphology of the tooth; hence the possibility of creating more apical contact points, inducing dental diastema closure, or reducing interdental spaces (Prato G P, Rotundo R, Cortellini P, Tinti C, Azzi R. Interdental papilla management: a review and classification of the therapeutic approaches. Int J Periodontics Restorative Dent 2004; 24(3):246-55). The possibility of recreating new and more favourable anatomical determinants, with the aim of inducing proliferative responses of soft tissues, also leads to aesthetic closure of the so-called black triangles. Importantly, the extent of the morphological modification of the tooth should be evaluated on the basis of the expected response of the soft tissues. It is therefore not considered a good practice to correct papilla-free interdental areas with a total reconstructive closure of the tooth. The resulting overcorrection, in fact, can induce unwanted tissue reactions.

Orthodontic treatment also modifies the anatomical determinants, but in this case, this is carried out with approaches that are altogether more physiological. Periodontal disease, together with problems of bone resorption and soft tissue recession, often leads to tooth displacement, especially in the anterior-superior area. When the support tissue is reduced and inflamed, the dental elements respond to functional forces with migration, inclination and flaring movements. This results in accentuation of interproximal tissue loss due to changes in the relationships between the anatomical determinants. After appropriate periodontal therapy, orthodontic treatment can therefore be useful for corrective purposes (Cardaropoli D, Re S, Corrente G, Abundo R. Reconstruction of the maxillary midline papilla following a combined orthodontic-periodontic treatment in adult periodontal patients. J Clin Periodontol 2004; 31(2):79-84; Cardaropoli D, Re S. Interdental papilla augmentation procedure following orthodontic treatment in a periodontal patient. J Periodontol 2005; 76(4):655-61; Melsen B, Agerbaek N, Markenstam G. Intrusion of incisors in adult patients with marginal bone loss. Am J Orthod Dentofacial Orthop 1989; 96(3):232-41). However, even orthodontic therapy has application limits. Regardless of the possible anchoring difficulties, obvious limitations, in fact, derive from the interarch relationships. For this reason, orthodontic therapy is often assisted, in the final stage, by conservative-prosthetic corrections.

In 1985, Shapiro described a procedure of periodic curettage of papillary tissues to stimulate their regrowth (Shapiro A. Regeneration of interdental papillae using periodic curettage. Int J Periodontics Restorative Dent 1985; 5(5):26-33). This simple manoeuvre, repeated every 15 days for 3 months, has proven to be capable of inducing soft tissue hyperplasia. However, this technique can be a plausible option only in the case of very aggressive periodontal disease, and anyway, positive results have not been obtained in all cases treated by the author.

In 2007, the proposal of injection of fibroblasts into interdental tissues was published (McGuire M K, Scheyer E T. A randomized, double-blind, placebo-controlled study to determine the safety and efficacy of cultured and expanded autologous fibroblast injections for the treatment of interdental papillary insufficiency associated with the papilla priming procedure. J Periodontol 2007; 78(1):4-17). The authors described a non-surgical method represented by the injection of successive quantities of autologous fibroblasts (FBL). The basis for this technicality lies in the trophic role attributed to these cells. FBLs, by producing collagen matrix and fibres, play an important role in healing and maintaining the health of the gum. It was therefore hypothesized that papilla regrowth could be promoted by using these cells. However, the peculiarity of the proposed technicality limits its applicability on an outpatient basis.

More recently, in addition to traditional therapies, less invasive treatments were proposed by infiltration of hyaluronic acid into the gum. Treatments are carried out by filling in the missing papillary tissue with fillers. This filling is carried out by using cross-linked hyaluronic acid, which acts as a filler, in order to create a volume and fill in the missing area of the papilla (Bertl K, Gotfredsen K, Jensen S S, Bruckmann C, Stavropoulos A. Can hyaluronan injections augment deficient papillae at implant-supported crowns in the anterior maxilla? A randomized controlled clinical trial with 6 months follow-up. Clin Oral Implants Res. 2017 September; 28(9):1054-1061(a)). Cross-linked hyaluronic acid, which creates a volumetric effect in the tissue where it is infiltrated, can even persist for many months, since degradation occurs very slowly. In fact, the molecule is protected from hyaluronidase degradation as a result of the chemical modification treatment performed by means of the cross-linking step. However, the results obtained are conflicting and not without the occurrence of adverse reactions (Bertl K, Gotfredsen K, Jensen S S, Bruckmann C, Stavropoulos A. Adverse reaction after hyaluronan injection for minimally invasive papilla volume augmentation. A report on two cases. Clin Oral Implants Res. 2017 July; 28(7):871-876)(b)).

The present invention is based on the acknowledgement that co-administration of hyaluronic acid and polynucleotides, preferably via the topical and/or infiltration route, exerts a positive effect both on periodontitis and the regeneration of the interdental papilla, showing a synergy between the components which allows the achievement of results that are superior to the administration of the components used separately.

The present inventors found that, in the context of periodontitis and diseases characterised by recession of the interdental papilla, such as for example the black triangle syndrome, the combination of polynucleotides and hyaluronic acid has anti-inflammatory, trophic and biostimulant effects in the infiltration areas. It was also found that the composition for use according to the present invention, comprising the combination of polynucleotides and hyaluronic acid, wherein the hyaluronic acid is preferably not cross-linked, is quickly reabsorbed. Therefore, compared to the cross-linked hyaluronic acid infiltrations described in the prior art (see for example EP 1525011 and the aforementioned publications by Bertl et al., 2017), it is essential to acknowledge that, in this case, this is not a mere filling treatment.

Thanks to the biochemical characteristics of polynucleotides and hyaluronic acid, in fact, the present invention allows the achievement of an anti-inflammatory effect and a physiological response of the tissues, which is capable of promoting a remarkable and spontaneous regeneration of the interdental papilla and an improvement of tissue trophism.

Further advantages are that the administration of the composition for use according to the present invention via the topical and infiltration routes is well tolerated and not too unpleasant for the patient, even without the use of local anaesthesia; the treatment is rapid and non-invasive; the treatment is repeatable and without discomfort for the everyday life of the patient, who can immediately resume a normal social life and a normal diet, contrary to what happens instead after surgery; no side effects were reported; the treatment is extremely effective and therefore patients are highly satisfied.

Therefore, the object of the invention is a composition with a synergistic effect, comprising polynucleotides and hyaluronic acid as the active ingredients, and the use thereof in the applications defined by the appended claims, which form an integral part of the description.

Hyaluronic acid (HA) is a non-sulphur-containing polymer belonging to the glycosaminoglycan family. It is ubiquitous in mammalian tissues, has an unbranched structure and consists of the orderly repetition of a disaccharide composed of glucuronic acid and N-acetylglucosamine.

The size of the HA varies, but exceeds one million Daltons, ranging from 3 to 9 million Daltons in some tissues. Initially, HA was isolated in 1934 by Mayer from the hyaloid substance of the vitreous body, hence the name, and for years HA has been considered a filling and structural substance with extraordinary hydrophilic capacity, with no particular biological role. Only in the last decade, attention has focused on the biological role of this polymer, especially after the discovery of tissue receptors for HA, such as CD44 and RHAMM. The knowledge of the role of HA as an active substance in more complex processes concerning tissue regeneration and repair has therefore broadened. Therefore, HA is no longer considered to be an inert molecule, but a molecule actively involved in important biological processes, such as cell migration and duplication, angiogenesis, and tissue growth and maturation.

Within the scope of the present invention, hyaluronic acid can be used as such or be variously salified, for example with sodium.

Typically, hyaluronic acid or a sodium salt thereof is used with a molecular weight of between 500 and 3000 kDaltons, preferably between 600 and 1600 kDaltons. Further molecular weights of hyaluronic acid usable within the scope of the present invention are 700 kDaltons, 800 kDaltons, 900 kDaltons, 1000 kDaltons, 1200 kDaltons, 1400 kDaltons, 1600 kDaltons, 1800 kDaltons, 2000 kDaltons, 2200 kDaltons, 2400 kDaltons, 2600 kDaltons, 2800 kDaltons.

Non-crosslinked hyaluronic acid is preferably used.

The term “Polynucleotides” is intended to indicate a fraction of nucleic acid polymer chains, preferably DNA, having a molecular weight distribution preferably in the range between 0.5 kDaltons and 3000 kDaltons, more preferably between 30 kDaltons and 2000 kDaltons, with a numerical average molecular weight, for example, of approximately 250 kDaltons. Said fraction of DNA polymer chains is preferably obtained by extraction from animal or plant natural sources, such as fish sperm or plants, by means of a process which preferably comprises a step of partial fragmentation of the DNA chains, in order to reduce the molecular weights thereof, and a step of partial depurination of the DNA, to eliminate the informational capacity thereof.

The preferred percentage of DNA depurination is preferably in the range of from 1% to 5% purine bases removed (i.e. apurinic sites) out of the total purine bases originally present. A preferred depurination percentage is between 2% and 2.5%. Further depurination percentages of polynucleotides usable within the scope of the present invention are 1.5%, 1.6%, 1.7%, 1.8%, 1.9%, 2.1%, 2.2%, 2.3%, 2.4%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%.

From the foregoing, it is obvious that the characteristics of the PNs are significantly different from those of conventional polynucleotides which have not been subjected to:
partial depurination,
sterilisation techniques for viruses and prions,
procedures for purification of the product which, when dry, exceeds 98% purity with quantities of proteins below 0.5% and a molecular weight range varying between 0.2 and 3000 kDaltons.

It is known and documented that PNs have the ability to induce mitosis in fibroblasts, endothelial cells and other cells. They have been used to stimulate tissue repair and in many experimental models have shown a remarkable ability to promote ex novo synthesis of extracellular matrix molecules.

In all wound-healing processes, the scientific literature confirmed that PNs have a positive effect on damage repair. PNs also have a remarkable ability to modulate the action of many inflammatory factors and interact with physiological growth factors, which makes these molecules very important and biologically very active in the growth of different cell lines and the secretory activity thereof.

The polynucleotides used in the present invention have a molecular weight distribution preferably in the range between 0.2 kDaltons and 3000 kDaltons, more preferably between 30 kDaltons and 2000 kDaltons, with a numerical average molecular weight of approximately 250 kDaltons, for example. The molecular weight distribution of the polynucleotides used in the scope of the present invention can also be included within the following ranges: 5-2500 kDaltons; 50-1500 kDaltons; 100-1000 kDaltons.

The composition according to the invention comprises:
hyaluronic acid as described above
polynucleotides (PNs) as described above.

For liquid formulations (for example formulations suitable for injection or topical application), for example, polyols are used in order to obtain a suitable osmolarity. A preferred polyol is mannitol in concentrations from 0 mg/ml to 50 mg/ml. To ensure an approximately neutral pH (7.0+/−1.0) a buffer system is used, preferably phosphate buffer.

The mannitol used can be sterile, pyrogen-free, pharmaceutical grade, for injectable use according to the European Pharmacopoeia.

The phosphate buffer used can be pharmaceutical grade, injectable, sterile, pyrogen-free.

In addition to liquid formulations, the PN+HA combination for use according to the present invention can also be used in the form of gel formulations known per se and commercially available, for example, as Nucliaskin® Oral care from Mastelli S.r.l.

Compositions used typically comprise:
polynucleotides from 1 mg/ml to 20 mg/ml, preferably from 5 mg/ml to 12 mg/ml,
hyaluronic acid from 1 mg/ml to 20 mg/ml, preferably from 5 mg/ml to 12 mg/ml, with polynucleotide/hyaluronic acid weight ratios within the range from 0.2:1 to 2:1.

### Example 1

Mannitol: 40 mg/ml

phosphate buffer: 1.3 mg/ml

hyaluronic acid: 7.5 mg/ml

PNs: 7.5 mg/ml

Water: 943.7 mg/ml

### Example 2

Mannitol: 40 mg/ml

phosphate buffer: 1.3 mg/ml

hyaluronic acid: 10 mg/ml

PNs 10 mg/ml

Water: 938.7 mg/ml

### Example 3

Mannitol: 40 mg/ml

phosphate buffer: 1.3 mg/ml

hyaluronic acid: 3.5 mg/ml

PNs 3.5 mg/ml

Water: 951.7 mg/ml

### Example 4

Mannitol: 40 mg/ml

phosphate buffer: 1.3 mg/ml

hyaluronic acid: 5 mg/ml

polynucleotides: 15 mg/ml

Water: 938.7 mg/ml

### Example 5

phosphate buffer: 1.3 mg/ml

hyaluronic acid: 3 mg/ml

PNs (respectively): 7.5, 11, 16 mg/ml

Water: 948.2; 944.7; 939.7 mg/ml

### Example 6

Mannitol: 40 mg/ml

Phosphate buffer: 1.3 mg/ml

Hyaluronic acid: 17 mg/ml

Polynucleotides: 3 mg/ml

Water: 938.7 mg/ml

The effectiveness of the compositions described above in the treatment of periodontitis has been determined by clinical trials on patients suffering from acute or chronic periodontitis. In these clinical trials, which are described in detail in the following experimental section, a commercial composition falling within the scope of the present invention (20 mg/ml Newest—class III medical device—CE 0373) was administered to the patients into the periodontal pockets and through local infiltration into the vestibule. The results obtained show that the administered composition was able to reduce the depth of the periodontal pocket in 94% of treated cases. A significant improvement was also observed in subjective parameters, such as burning, pain, discomfort, bleeding, swelling and redness.

The effectiveness of the compositions described above in the regeneration of the interdental papilla was determined by clinical trials on patients who exhibited at least one site with papilla loss in the aesthetic area, a papillary defect commonly referred to as the “black triangle syndrome”. In these clinical trials, which are described in detail in the following experimental section, a commercial composition falling within the scope of the present invention (20 mg/ml Newest—class III medical device—CE 0373) was administered to the patients through infiltration into the papilla and the surrounding area (vestibule). The results obtained show that the administered composition was able to induce an improvement in the clinical picture of the papilla in 100% of treated cases. Furthermore, as shown by an extended clinical study, administration by infiltration of the aforementioned commercial composition did not cause the side or adverse effects described in the state of the art (in particular in Bertl K. et al., Clin Oral Implants Res. 2017 September; 28(9):1054-1061(a) and Bertl K et al., Clin Oral Implants Res. 2017 July; 28(7):871-876)(b)), presumably thanks to the combination of hyaluronic acid with polynucleotides.

The synergistic effect of the compositions comprising PNs and HA was determined by in vitro tests, described in detail in the following experimental section, aimed at determining the effect on the growth of human gingival fibroblast cells and on protein matrix deposition. The results obtained show that, while the effect of HA alone is modest, its combination with PNs results in increased effectiveness of the latter. It can therefore be concluded that by combining HA and PNs a synergistic effect is obtained at the gingival fibroblasts in terms of both cell regeneration and protein matrix deposition. This, in particular, may explain the special effectiveness of the HA and PN combination in the treatment of the black triangle syndrome and in periodontal therapies.

Lastly, studies were carried out for assessing local inflammation and the presence of metalloproteinases at periodontal pockets (assessed through the harvesting of crevicular fluid and measurement of alpha-2-macroglobulin and MMP-9 markers analysed by the ELISA method), in order to verify the modulation action of the product on inflammatory cytokines, as well as the local tolerability of a composition comprising the combination of PNs and HA.

In the study, 25 patients suffering from periodontal disease with at least 2 periodontal pockets were evaluated. All patients were treated with a normal hygiene and prophylaxis standard therapy, and in addition, the test MD was used in one of the two pockets, in a randomized way, through a single application in the periodontal pocket.

The results show that, in the case of alpha-2-macroglobulin, 100% of periodontal pockets treated with the test MD exhibited an improvement, that is a 68% improvement, whereas, in the untreated group, 1 pocket worsened, and the average improvement was 63%.

As for the MMP-9 parameter, the results show that periodontal pockets treated with the test MD exhibited a 69% improvement, whereas 6 untreated pockets worsened (24%) and the average improvement in the untreated pockets was significantly lower, that is 49%. These results are particularly significant also because each patient serves as a self-control, i.e., the patient has both a treated pocket and an untreated pocket, thus eliminating any possibility of interference due to individual variability.

In the laboratory, therefore with objective and blind parameters, it also appears that treatment with polynucleotides+hyaluronic acid brings about an improvement compared to the standard hygiene and prophylaxis therapy, in particular by modulating and reducing parameters related to inflammation and by reducing MMP9 activity. Also underlining the high tolerability and biocompatibility of the device.

### EXPERIMENTAL SECTION

1. Periodontal Therapy—Clinical Study

15 patients aged between 30 and 83 were treated in the study, including 2 men and 13 women suffering from acute periodontitis (3 patients) or chronic periodontitis (12 patients). Periodontitis is defined as periodontal tissue inflammation, which causes loss of attachment of the teeth to the alveoli, which results in formation of periodontal pockets, loose teeth, bleeding gums, abscesses and suppurations, ending in the loss of one or more teeth. 12/15 patients had disease familiarity when they entered the study, 6/15 were smokers. Each patient underwent hygiene and prophylaxis as per guidelines and was then treated with a polynucleotide and hyaluronic acid gel (20 mg/ml Newest—class III medical device—CE 0373).

!TABLE 1
Patients: general data

| | |
|---|---|
| Total number of patients | 15 |
| age | min 30-max 83 |
| M/F | 2 M/13 F |
| acute periodontitis | 3 (out of 15) |
| chronic periodontitis | 12 (out of 15) |
| smokers | 6 (out of 15) |
| familiarity | 12 (out of 15) |

Materials and Methods:

Each patient underwent a specific therapeutic program consisting of 3 main phases. One visit was scheduled for each phase, for a total of 3 visits. The program is described in detail below:
1) Pre-Treatment Phase: hygiene and prophylaxis sessions are scheduled and performed (for example, manual and ultrasonic therapy, aimed at decreasing the presence of plaque, tartar and therefore bacteria).

An average of 2-3 20-30-minute sessions of hygiene and prophylaxis were carried out for each patient on a monthly basis. Each patient underwent the first visit (V1) during this phase.

At V1, patients were clinically evaluated to record subjective parameters (symptoms reported by the patient) and objective parameters (signs detected by the physician).
2) Treatment phase with the test medical device: once clean, the patients were administered via the topical and infiltration routes with the polynucleotide and hyaluronic acid gel (20 mg/ml Newest); this phase was divided into several sessions:
The first session involved topical application into the periodontal pockets and local infiltrations into the vestibule.
The subsequent sessions, carried out on a monthly basis, only involved local infiltrations into the vestibule.

Each patient underwent the second visit (V2) during this phase. At V2, subjective and objective parameters were evaluated again.
3) Maintenance phase: a session of hygiene and local infiltrations into the vestibule with the polynucleotide and hyaluronic acid gel (20 mg/ml Newest) is carried out every 3-6 months.

Each patient underwent the last visit (V3) of the study during this phase. At V3, subjective and objective parameters were evaluated again, in order to evaluate the clinical course of the disease compared to the initial (basal) situation.

Application into the periodontal pockets was carried out by using a blunt tip applicator.

Local infiltrations into the vestibule were carried out with a 30 G needle, at a distance of 0.5-1 cm, after appropriate local disinfection.

Results:

At the end of the study, 27% of treated cases exhibited a pocket depth reduction of 2 mm, whereas 67% of cases exhibited a reduction of 1 mm. Only in 6% of cases the pocket depth remained unchanged.

Burning, pain, discomfort, bleeding, swelling and redness were the subjective parameters assessed during the study at V1 (pre-treatment phase), V2 (treatment phase) and V3 (maintenance phase). All the tested parameters showed an improvement in the starting conditions. In detail:

Pain reduction, assessed by using a VAS scale with a score of 0-10, averaged 67.3%. In fact, the average value ranged from 3.7 at V1 to 1.2 at V3, with an average reduction of 2.5 points.

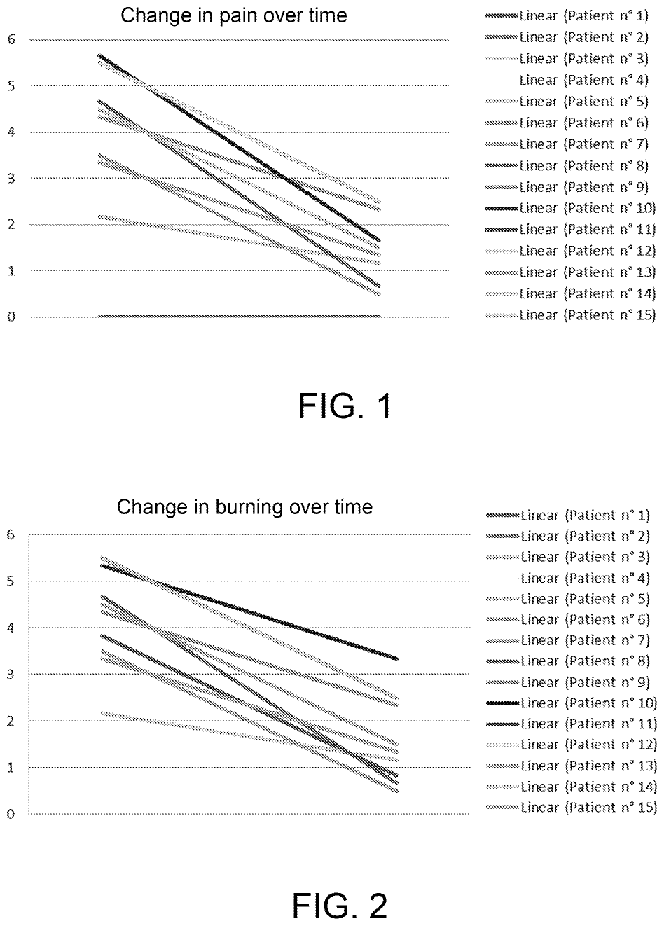

The graph in FIG. 1 shows the reduction in the sensation of pain perceived by each patient from visit V1 to visit V3.

The reduction in burning, assessed by using a VAS scale with a score of 0-10, averaged 64.4%. In fact, the average value ranged from 3.9 at V1 to 1.4 at V3, with an average reduction of 2.5 points.

The graph in FIG. 2 shows the reduction in the sensation of burning perceived by each patient from visit V1 to visit V3.

The reduction in discomfort, assessed by using a VAS scale with a score of 0-10, averaged 63.6%. In fact, the average value ranged from 3.7 at V1 to 1.3 at V3, with an average reduction of 2.3 points.

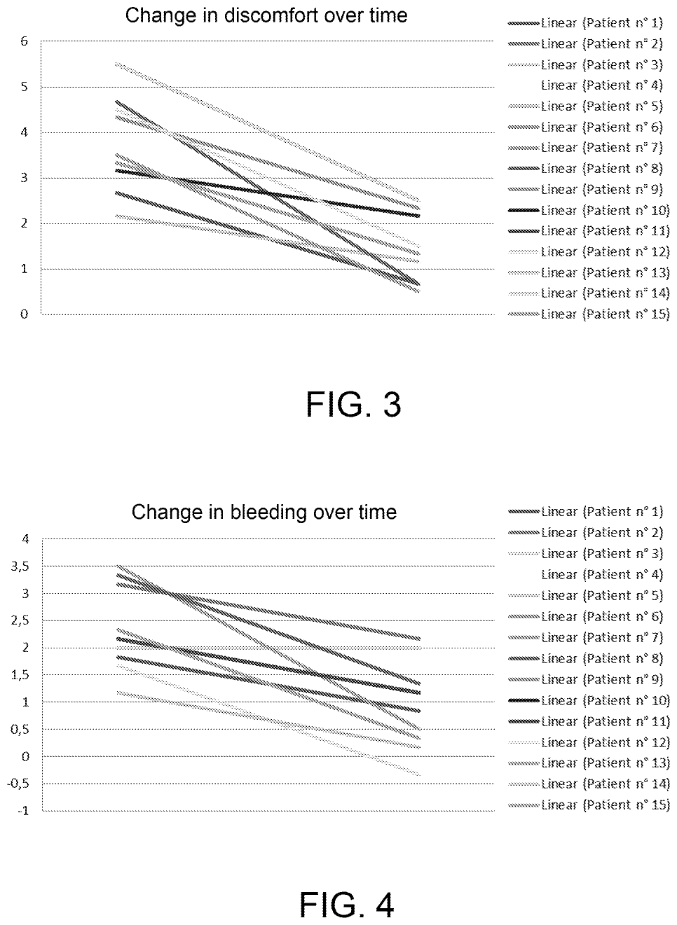

The graph in FIG. 3 shows the reduction in the sensation of discomfort perceived by each patient from visit V1 to visit V3.

The reduction in bleeding, assessed on a scale with a score from 0-3, where 0 indicated no bleeding and 3 heavy bleeding, was on average 64.7%, in fact, the average value ranged from 2.3 at V1 to 0.8 at V3, with an average reduction of 1.5 points.

The graph in FIG. 4 shows the reduction in bleeding obtained for each patient from visit V1 to visit V3.

In conclusion, the treatment of chronic and generalized periodontal diseases (11/15 cases treated) with the gel containing the PN+HA combination gave excellent results in terms of disappearance of swelling and redness. There was also an improvement of over 60% in signs such as bleeding and symptoms such as burning, pain and discomfort. There was also an objective improvement in terms of reduction of the pocket depth.

Therefore, in the dental field, treatment with the gel containing the PN+HA combination represents a valuable and innovative therapeutic choice associated with hygiene and prophylaxis sessions, to improve and enhance the response to the classic hygiene and prophylaxis therapy.

2. Clinical Study of Papilla Regeneration (Black Triangle Syndrome)

10 patients with an average age of 52 years, including 7 men and 3 women, were treated in the study. Two of the patients at the start of the study were smokers. In all cases, the patients exhibited at least one site with papilla loss in the aesthetic area, a papillary defect commonly referred to as the “black triangle syndrome”.

TABLE 2
Patients: general data

| | |
|---|---|
| Total number of patients | 10 |
| average age | 52 |
| M/F | 7 M/3 F |
| smokers | 2 (out of 10) |

Materials and Methods:

Each patient underwent a specific therapeutic program consisting of 2 main phases. One visit was scheduled for each phase during the study and one follow-up visit was scheduled after the treatment phase, for a total of 3 visits. The program is described in detail below:
1) Pre-Treatment Phase: The phase in which hygiene and prophylaxis sessions were scheduled and performed (for example, manual and ultrasonic therapy, aimed at decreasing the presence of plaque, tartar and therefore bacteria). All patients were instructed to perform mechanical cleaning at home by using an ultra-soft toothbrush and a brushing technique with movements in the apical-coronal direction.

An average of 2-3 20-30-minute sessions of hygiene and prophylaxis were carried out for each patient on a monthly basis. Each patient underwent the first visit (V1) during this phase.

Photographs were taken at V1 and the physician's clinical evaluation and patient satisfaction were recorded.
2) Treatment phase with the test medical device: At least one month after stabilization of the clinical picture following the hygiene and prophylaxis sessions, the patients were administered with the polynucleotide and hyaluronic acid gel (20 mg/ml Newest).

After disinfection with mouth rinses and local disinfectant at the infiltration site, the affected area was treated by infiltrating the papilla and the surrounding area (vestibule) with small doses of product, generally less than 0.1-0.2 ml. Local infiltrations into the vestibule were carried out with a 30 G needle, at a distance of 0.5-1 cm.

The treatment was repeated three to six weeks apart for a total of 3-10 infiltrations.

Each patient underwent the second visit (V2) during this phase. At V2, the effects were documented with photographs, compared with those taken before treatment and by filling in a data collection form with recording of the physician's clinical evaluation and patient satisfaction.

Results:

Patients' subjective evaluations were very positive both from the point of view of pain and compliance with the treatment and of satisfaction with said treatment, with the following results.

The clinical results were very encouraging. Evaluation based on a quartile scale showed improvement in 100% of patients. All cases showed a clinical improvement of the papilla, although not all of them showed complete healing.

On average, regrowth started to appear after the second or third session, although in some cases regrowth began after the seventh or eighth session.

As regards the objective evaluation of the papillae, all patients showed improvement in the papillary structure, such as to change their classification:
in three cases, class III papilla became class I papilla
in six cases, class II papilla became class I papilla
in one case, class I papilla became a Normal papilla (the papilla fills the interdental space again up to the point of contact).

In particular, it should be underlined that the improvement of the papilla occurred very quickly: all patients already exhibited an initial regeneration of the papilla after one week and this regenerative process continued over time. In fact, the sites were checked in a follow-up and the improvement that took place after the applications, one month after the last treatment, was still present. In no case was there a recurrence.

In conclusion, a trophic/regenerative effect of polynucleotides, which were only locally infiltrated, was shown for the first time in the treatment of the black triangle syndrome, a disease hitherto treated with invasive methods such as orthodontic, restorative and surgical interventions, which however yielded poor clinical results.

3. In Vitro Study Gingival Fibroblasts

Human gingival fibroblasts were cultured on growth medium (Dulbecco's Modified Eagle Medium) supplemented with 10% bovine foetal serum (BFS). Cells were seeded at a density of 10,000/cm². 24 hours after seeding the cells were incubated under the conditions shown in Table 3, again prepared in DMEM+10% BFS.

Cells were counted and proteins were quantified after 3 days of treatment.

The number of cells was measured with a particle counter. Proteins were quantified by spectrophotometric reading of stained protein lysates by using a modification of Lowry's method.

Table 3 shows the concentrations of the tested compositions and the values relating to cell growth and protein content, expressed as % of the control. The control is the fibroblast cell culture not subjected to any treatment, measured at the same time point.

TABLE 3

| | % gingival | % gingival |
|---|---|---|
| | fibroblasts vs | fibroblasts vs |
| Product | control: cell growth | control: proteins |
| pn 50 μg/ml | 123 | 124 |
| pn 100 μg/ml | 127 | 129 |
| pn 300 μg/ml | 128 | 129 |
| ha 50 μg/ml | 102 | 103 |
| ha 100 μg/ml | 105 | 104 |
| ha 300 μg/ml | 106 | 104 |
| pn + ha 50 μg/ml | 127 | 130 |
| pn + ha 100 μg/ml | 131 | 130 |
| pn + ha 300 μg/ml | 132 | 133 |
| control | 100 | 100 |

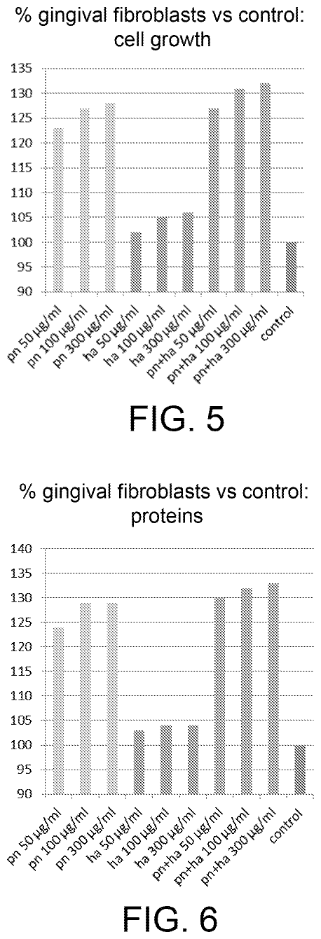

Cell growth and protein quantification results are also shown in FIGS. 5 and 6.

The results show that hyaluronic acid does not change its action as its concentration increases, constantly maintaining a mild effect both in terms of protein expression and cell growth. Instead, it is clear that polynucleotides increase their effect as the dose increases. Compared to the individual components, the effect obtained with the mixture—which, for each total concentration, consisted of 50% hyaluronic acid and 50% polynucleotides—was consistently superior to the effect of the PNs used alone and the HA used alone.

Accordingly, bearing in mind the low effect of hyaluronic acid alone, it is obvious that the presence of the latter is important for increasing the effect of the PNs, which are the most active component in cell growth and protein expression. It can be concluded that by combining HA and PNs a synergistic effect is obtained at the gingival fibroblasts in terms of both cell regeneration and protein matrix deposition. This, in particular, may explain the special effectiveness of the HA and PN combination in the treatment of the black triangle syndrome and in periodontal therapies.

4. In Vivo Study of the Crevicular Fluid

Treatment of periodontitis first of all involves controlling bacteria and inflammation, and immediately afterwards, the subsequent step of regenerating the lost, hard and soft periodontal tissues becomes essential, in order to restore tooth attachment and eliminate gingival and bone pockets.

ODONTO-PNHA is a class DI Medical Device manufactured by Mastelli Srl—Sanremo (IM). It is an original combination of polynucleotides and hyaluronic acid for application in periodontal pockets.

The main purpose of the clinical study with Odonto—PNHA is to evaluate the efficacy and safety of a medical device in non-surgical periodontal therapy. The experimental hypothesis is that the addition of ODONTO-PNHA can help obtain periodontal attachment gain in the periodontal pockets, in order to avoid a surgical procedure.

To evaluate this, a total of 50 patients were recruited: each patient provided a test site (treated with ODONTO-PNHA) and a control site (untreated). Patients will be followed up for 12 months after treatment. The study is performed in a single clinical centre, at the Department of Periodontology, Universitá La Sapienza.

Primary objective: evaluation of ODONTO-PNHA's performance on Probing Pocket Depth (PPD), Marginal Bleeding Index (MB), Modified Plaque Index (mPLI), pain, Investigator Global Satisfaction with the usability of the device and Patient Global Satisfaction with the performance of the device (5-point Likert scale). Tolerability and safety assessment.

Objective of the biochemical analysis: Evaluation of the effects of ODONTO-PNHA on the inflammatory state of the tissues around the tooth in 25 subjects. This was assessed through harvesting the crevicular fluid and assessing alpha 2 macroglobulin (a2M) and metalloprotease-9 (MMP-9) in the fluid at baseline and at week 6.

The crevicular fluid samples, adsorbed on methylcellulose strips, were processed and assayed with the ELISA method.

Matrix metalloproteinases are an important group of proteinases related to collagen degradation during the destructive process of periodontal disease, which can be measured in the gingival crevicular fluid. Alpha 2 macroglobulins are plasma proteins that participate in the regulation of blood clotting and immune response and increase in the presence of a chronic infection. 25 patients suffering from periodontal disease and recruited in the ODONTO-PNHA-01 clinical study underwent two takings of crevicular fluid, one taking from the periodontal pocket treated with the test MD and one from an untreated pocket.

The takings were performed at baseline (Visit 1) and at week 6 (Visit 2). The total 4 samples were assessed for the presence and concentration of inflammation markers.

Results

After 6 weeks, the concentrations of the markers, both in the pocket treated with the polynucleotide and hyaluronic acid MD and in the untreated pocket, were significantly lower compared to week 0, showing a general improvement in periodontal disease, thanks to the standard hygiene and prophylaxis therapy.

Analysis of data from the test MD-treated and untreated periodontal pockets showed a further difference, described below, to the advantage of patients who had received treatment with the test MD.

In the treated pockets, the average alpha 2 macroglobulin value was 1898.71 at time 0 and 605.90 after 6 weeks, with an average reduction of 1292.81 corresponding to 68.09%. Out of 25 treated pockets, 19 (76%) showed an improvement of more than 50% (Table 4).

In the untreated pockets, the average alpha 2 macroglobulin value was 1665 at time 0 and 601 after 6 weeks, with an average reduction of 1064 corresponding to 63.91%. Out of 25 treated pockets, 17 (68%) showed an improvement of more than 50% (Table 4).

TABLE 4

| | TREATED | CONTROLS |
|---|---|---|
| Total number | 25 | 25 |
| of patients | | |
| Start of the study | 1898.7 | 1665.3 |
| 6 weeks | 605.9 | 601.1 |
| Delta | 1292.8 | 1064.3 |
| % Improvement | 68.1% | 63.9% |
| compared to | | |
| baseline | | |
| No. improved | 19 | 17 |
| patients > 50% | | |

An even greater difference between treated and controls was found in the MMP-9 parameter. In the treated pockets, the average MMP-9 value was 234.9 at time 0 and 72.9 after 6 weeks, with an average reduction of 162.00 corresponding to 68.96%. Out of 25 treated pockets, 18 (72%) showed an improvement of more than 50% (Table 5).

In the untreated pockets, the average MMP-9 value was 173.4 at time 0 and 87.4 after 6 weeks, with an average reduction of 86 corresponding to 49.58%. Out of 25 treated pockets, only 9 (36%) showed an improvement of more than 50% (Table 5).

TABLE 5

| | TREATED | CONTROLS |
|---|---|---|
| Total number | 25 | 25 |
| of patients | | |
| Start of the study | 234.9 | 173.4 |
| 6 weeks | 72.9 | 87.4 |
| Delta | 162.0 | 86.0 |
| % Improvement | 68.96% | 49.58% |
| compared to | | |
| baseline | | |
| No. improved | 18 out of 25 (72%) | 9 out of 25 (36%) |
| patients > 50% | | |
| No. worsened patients | 2 (8%) | 6 (24%) |

The results show that, in the case of alpha 2 macroglobulin, all treated patients showed an improvement, whereas in the untreated group, 1 patient worsened; in the case of the MMP-9 parameter, all patients except 1 showed an improvement, whereas in the untreated group, 6 patients showed a worsening.

In general, improvement was greater in the test MD-treated patients and this improvement was particularly clear for the MMP-9 parameter.

These results are particularly significant also because each patient serves as a self-control, i.e., the patient has both a treated pocket and an untreated pocket, thus eliminating any possibility of interference due to individual variability.

### CONCLUSION

In the laboratory, therefore with objective and blind parameters, it also appears that treatment with polynucleotides+hyaluronic acid brings about an improvement compared to the standard hygiene and prophylaxis therapy, in particular by modulating and reducing parameters related to inflammation and by reducing MMP9 activity.

Therefore, the preliminary laboratory results illustrated above support the experimental hypothesis that the PN+HA combination can help obtain periodontal attachment gain in periodontal pockets, in order to avoid surgical procedure.

1. A method of treating regeneration of interdental papilla or a therapeutic and/or cosmetic treatment of recession of interincisal papilla (black triangle syndrome) or a therapeutic and/or cosmetic treatment of periodontitis in patients in need thereof with a composition comprising hyaluronic acid and a mixture of polynucleotides extracted from a natural source, said method comprising
administering to said patients a pharmaceutical effective amount of said composition.

2. The method according to claim 1, wherein said periodontitis is acute periodontitis or chronic periodontitis.

3. The method according to claim 1, wherein said therapeutic and/or cosmetic treatment results in reduction in the depth of the periodontal pockets and/or in pain reduction and/or in stinging reduction and/or in discomfort reduction and/or in bleeding reduction.

4. The method according to claim 1, wherein said therapeutic and/or cosmetic treatment causes the regeneration of the papilla.

5. The method according to claim 1, wherein said therapeutic and/or cosmetic treatment results in increased proliferation of gingival fibroblasts and increased deposition of a protein matrix.

6. The method according to claim 1, wherein the treatment is carried out by topical application and/or local infiltration.

7. The method according to claim 1, wherein the treatment is carried out by topical application in the periodontal pockets.

8. The method according to claim 1, wherein the treatment is carried out by local gingival infiltration in the papilla and/or in the vestibule.

9. The method according to claim 1, wherein the pharmaceutical effective amount of the composition comprises from 1 mg/ml to 20 mg/ml of hyaluronic acid and from 1 mg/ml to 20 mg/ml of said mixture of polynucleotides.

10. The method according to claim 9, wherein the composition comprises from 5 mg/ml to 12 mg/ml of hyaluronic acid and from 5 mg/ml to 12 mg/ml of said mixture of polynucleotides.

11. The method according to claim 9, wherein the polynucleotides and the hyaluronic acid are in a weight ratio of from 0.2:1 to 2:1.

12. The method according to claim 1, wherein said mixture of polynucleotides is partially depurinated.

13. The method according to claim 12, wherein said mixture of polynucleotides has a depurination ratio ranging from 1% to 5% purine bases removed out of the total purine bases originally present.

14. The method according to claim 1, wherein said mixture of polynucleotides is a fraction of nucleic acid polymer chains having a molecular weight distribution ranging from 0.5 kDaltons to 3000 kDaltons.

15. The method according to claim 1, wherein said mixture of polynucleotides is obtainable by extraction from a natural, animal or plant source, by a process comprising a step of partial fragmentation of the polynucleotide nucleic acid chains and a step of partial depurination of the nucleic acid.

16. The method according to claim 1, wherein the hyaluronic acid is not cross-linked.

17. The method according to claim 1, comprising hyaluronic acid with a molecular weight of between 500 kDaltons and 3000 kDaltons.

18. The method according to claim 1, wherein said composition is in liquid form, free of physiological saline solution, in an aqueous solvent comprising a pharmaceutically acceptable polyol.

19. The method according to claim 18, wherein said composition has an osmolarity between 10 m.o.s./l and 500 m.o.s./l.

20. The method according to claim 1, wherein said composition is in a dosage form suitable for topical application or local infiltration.
